(19) United States
(12) Patent Application Publication
(10) Pub. No.: US 2021/0174893 A1
(43) Pub. Date: Jun. 10, 2021

(54) PROTEIN STRUCTURE PREDICTION

(71) Applicant: Homodeus, Inc., Guilford, CT (US)

(72) Inventors: Benjamin Rosenbluth, Hamden, CT (US); Brian Reed, Madison, CT (US); Jonathan M. Rothberg, Guilford, CT (US)

(73) Assignee: Homodeus, Inc., Guilford, CT (US)

(21) Appl. No.: 17/118,405

(22) Filed: Dec. 10, 2020

Related U.S. Application Data
(60) Provisional application No. 62/946,283, filed on Dec. 10, 2019.

(51) Int. Cl. G16B 15/20; G01N 21/64; G16B 35/00

(52) U.S. Cl. CPC G16B 15/20; G16B 35/00; G01N 21/6428

(57) ABSTRACT

The present disclosure provides, in some aspects, methods for using FRET-based distance measurements to refine and constrain protein structure prediction algorithms.

### RELATED APPLICATION

This application claims the benefit under 35 U.S.C. § 119(e) of U.S. provisional application number 62/946,283, filed Dec. 10, 2019, which is incorporated by reference herein in its entirety.

### BACKGROUND

Protein engineering is a process of developing useful or valuable proteins, or of modifying a protein by altering its chemistry, usually to improve its function for a particular application. Proteins are biological machines with many industrial and medical applications; proteins are used in detergents, cosmetics, bioremediation, industrial-scale reactions, life science research, and the pharmaceutical industry, with many modern drugs derived from engineered recombinant proteins.

Solving protein structures is a fundamental step in engineering proteins. The primary goal is to identify target amino acid residues that are most likely to influence protein function. Mutation of these amino acids leads to the creation of libraries of protein variants, some of which will have enhanced properties. Identifying these key amino acids is an important step for rational design of proteins and some variations of directed evolution including site-directed mutagenesis techniques. These variants are then expressed and tested for activity.

### SUMMARY

The present disclosure provides methods for determining the three-dimensional structure of a protein. The inventors recognized that combining a computer-implemented protein structure prediction algorithm with at least one empirically measured distance between two amino acid residues using in vitro experiments could enable accurate determination of three-dimensional protein structures at low cost and with minimal time. A first prediction of a protein structure in silico can be used to identify pairs of amino acids for analysis in an in vitro biochemical experiment. The in vitro biochemical experiment is then designed to empirically measure distances between the two amino acids in solution. These measured distances can be further utilized to constrain and refine the protein structure prediction algorithm in order to generate a second-generation prediction of the structure of the protein.

Some aspects of the present disclosure provides methods comprising: (i) performing in silico a three-dimensional structure prediction of a protein using a structure prediction algorithm; (ii) identifying in silico at least one pair of solvent-exposed amino acids in the protein based on at least one algorithm-predicted factor; (iii) labeling in vitro the at least one pair of amino acids in at least one recombinant copy of the protein such that a fluorescence resonance energy transfer (FRET) donor is attached to the first amino acid of the pair and a FRET acceptor is attached to the second amino acid of the pair; (iv) collecting in vitro distance measurements between the two amino acids of the at least one pair using FRET; and (v) constraining the structure prediction algorithm using the collected distance measurements. In some embodiments, the at least one algorithm-predicted factor that allows for identification of the at least one pair of solvent-exposed amino acids is variance in the spatial distance between the two amino acids of the at least one pair, the relative importance of the distance between the two amino acids in the structure prediction algorithm and/or the structural sensitivity of the pair.

Other aspects of the present disclosure provide computer-implemented methods comprising: performing in silico a three-dimensional structure prediction of a protein using a structure prediction algorithm; identifying in silico at least one pair of solvent-exposed amino acids in the protein based on algorithm-predicted factors (e.g., variance in the spatial distance between the two amino acids of the at least one pair); and constraining the structure prediction algorithm using distance measurements collected in vitro between amino acids of the at least one pair of amino acids present in a recombinant copy of the protein using fluorescence resonance energy transfer (FRET), wherein a FRET donor is attached to one amino acid of the pair and a FRET acceptor is attached to the other amino acid of the pair.

Yet other aspects of the present disclosure provide a computer readable medium on which is stored a computer program which, when implemented by a computer processor, causes the processor to: perform in silico a three-dimensional structure prediction of a protein using a structure prediction algorithm; identify in silico at least one pair of solvent-exposed amino acids in the protein based on algorithm-predicted factors (e.g., variance in the spatial distance between the two amino acids of the at least one pair); and constrain the structure prediction algorithm using distance measurements collected in vitro between amino acids of the at least one pair of amino acids present in a recombinant copy of the protein using fluorescence resonance energy transfer (FRET), wherein a FRET donor is attached to one amino acid of the pair and a FRET acceptor is attached to the other amino acid of the pair.

In some embodiments, the methods further comprise (vi) performing in silico a three-dimensional structure prediction of a protein using the constrained structure prediction algorithm, and optionally further repeating, at least 1, 2, 3, or more times, each of (ii) to (vi).

In some embodiments, the pair of amino acids are separated based on the primary structure of the protein by at least five amino acids.

In some embodiments, (i) comprises performing in silico a three-dimensional structure prediction of a protein using a structure prediction algorithm and generating a probabilistic matrix or distogram of the distances between each combination of two amino acids in the protein.

In some embodiments, (ii) comprises determining the algorithm-predicted variance in the spatial distance between every combination of two solvent-exposed amino acids and rank-ordering every combination of two solvent-exposed amino acids based on algorithm-predicted factors, optionally wherein the at least one pair of amino acids is identified as having the largest algorithm-predicted variance in spatial distance.

In some embodiments, in (ii), the algorithm-predicted variance in the spatial distance between the two amino acids comprises a k-value of between 1 and 100.

In some embodiments, the methods comprise: (i) performing in silico a three-dimensional structure prediction of a protein using a structure prediction algorithm; (ii) identifying in silico 2, 3, 4, 5, or more pairs of solvent-exposed amino acids in the protein based on algorithm-predicted variance in the spatial distance between the two amino acids of each pair; (iii) labeling in vitro each pair of amino acids in a recombinant copy of the protein such that a fluorescence resonance energy transfer (FRET) donor is attached to the first amino acid of each pair and a FRET acceptor is attached to the second amino acid of each pair, wherein each pair of amino acids is labeled in a different recombinant copy of the protein; (iv) collecting in vitro distance measurements between the two amino acids of each pair using FRET; and (v) constraining the structure prediction algorithm using the collected distance measurements.

In some embodiments, in (iii), each different recombinant copy of the protein comprises a unique molecular identifier or barcode sequence.

In some embodiments, in (iii), each different recombinant copy of the protein is placed into an individual well of a multi-well plate or an individual chamber of a zero-mode waveguide.

In some embodiments, each different recombinant copy of the protein is attached to the bottom of an individual well of a multi-well plate or an individual chamber of a zero-mode waveguide, optionally wherein each different recombinant copy of the protein is attached via a biotin-streptavidin linkage.

In some embodiments, one of the amino acids of the at least one pair is a cysteine, a lysine, or a non-natural amino acid, optionally wherein the non-natural amino acid is p-azido-L-phenylalanine.

In some embodiments, the FRET acceptor and FRET donor are organic dyes, fluorescent proteins, or quantum dots. For example, the fluorescent proteins may be cyan fluorescent proteins (CFPs) and yellow fluorescent proteins (YFPs); green fluorescent proteins (GFPs) and red fluorescent proteins (RFPs); or far-red fluorescent proteins (FFPs) and infrared fluorescent proteins (IFPs).

In some embodiments, the collecting in (iv) involves total internal reflection fluorescence, fluorescence lifetime imaging microscopy, or zero-mode waveguide sensing. In some embodiments, the collecting in (iv) is done using single-molecule methods.

In some embodiments, the at least one recombinant copy of the protein is barcoded. In some embodiments, the at least one recombinant copy of the protein is barcoded with a unique molecular identifier, optionally a nucleic acid-based or peptide-based unique molecular identifier.

### BRIEF DESCRIPTION OF THE DRAWINGS

FIG. 1 is a flow diagram of the steps of an illustrative process for performing the methods of the present disclosure.

FIG. 2 is a schematic showing FRET pairs on protein structures. Multiple pairs of solvent-exposed amino acids (typically estimated to be 2-10 nanometers apart) can be selected chosen for each variant. Each pair of amino acids is labeled with FRET dye molecules on a different protein to reduce experimental cross-talk and eliminate background uncertainty.

FIG. 3 is a schematic showing that, when 1:1 mixture of two FRET dye molecules (1:1 mixture of a FRET donor and a FRET acceptor) is conjugated to two exposed amino acid residues (e.g., two cysteines), there is a maximum theoretical labeling efficiency of 50% (i.e., 50% of labeled protein will have the correct pairing of FRET donor on one amino acid of the pair and FRET acceptor on the second amino acid of the pair).

FIG. 4 is a schematic showing the process of collecting distance measurements between several pairs of amino acids using FRET and then aggregating that distance measurement data into a distogram matrix. The data in the distogram matrix can then be used to constrain and refine the protein structure prediction model.

FIG. 5 is a flow diagram of an exemplary process labeling a protein with a non-natural amino acid.

FIG. 6 is a schematic showing a zero-mode waveguide apparatus containing multiple proteins having different pairs of amino acids labeled with FRET dyes. Each protein is conjugated via a streptavidin-biotin linker to the surface of an individual chamber of the zero-mode waveguide apparatus to enable collection of distance measurements between each of the different pairs of amino acids using FRET simultaneously.

FIG. 7 is a block diagram of an illustrative implementation of a computer system for determining protein structure.

FIG. 8 is a schematic of a protein structure prediction model.

FIG. 9 is a schematic of refined components of a protein structure prediction model.

FIG. 10 is a schematic of a generative model.

FIG. 11 is a schematic showing a series of distance matrix outputs capturing the structure of the target protein, relative to random initialization.

FIG. 12 is a schematic showing optimization of a genetic algorithm.

FIG. 13 is a schematic showing predicted structure outcomes following use of a genetic algorithm.

FIG. 14 is a schematic showing a framework for assessing the quality of a prediction produced by an algorithm.

FIGS. 15A-15D show a schematic showing built-in visualization allowed by a protein structure prediction algorithm.

FIG. 16 is a schematic showing predicted structure from a protein structure prediction algorithm compared to the true ground-state structure.

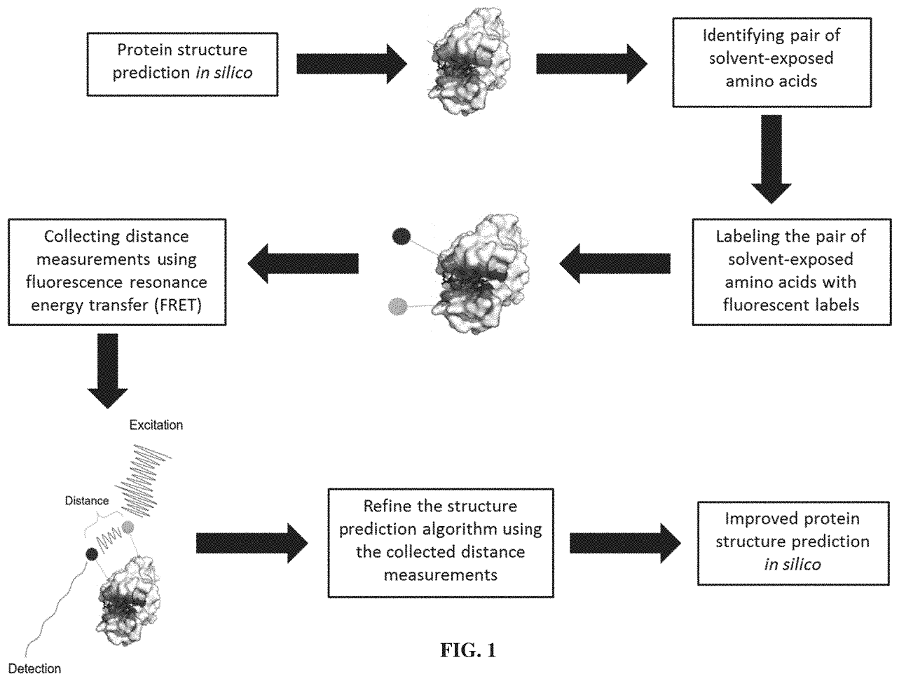

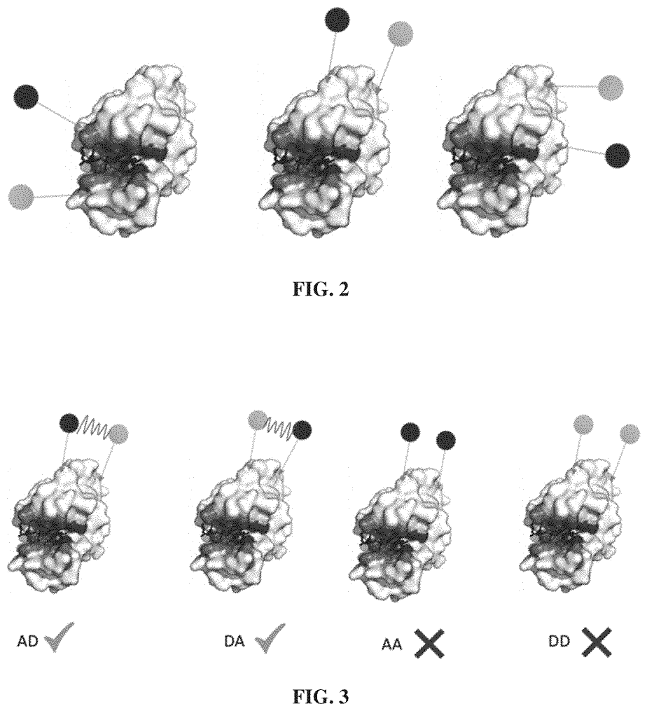

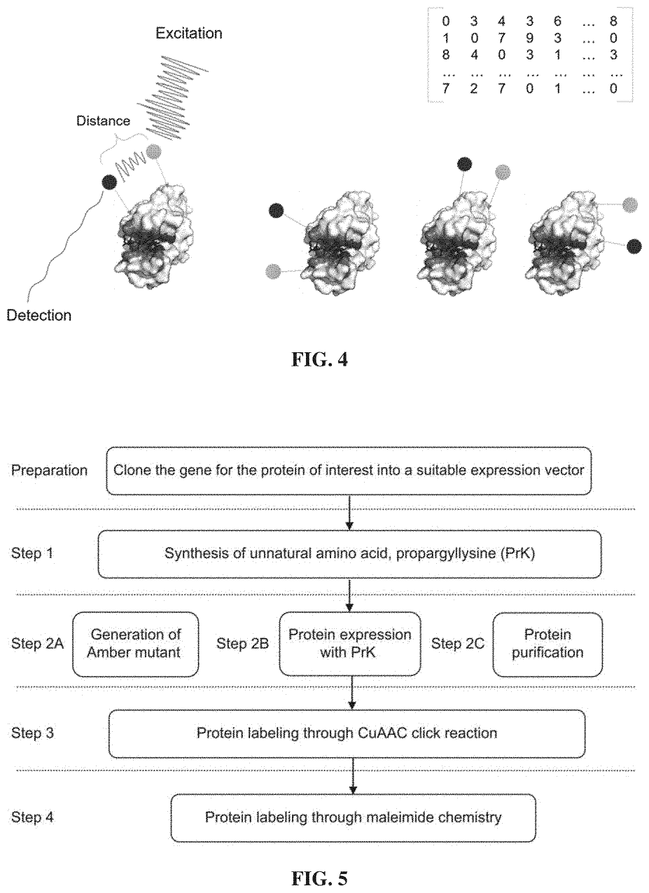

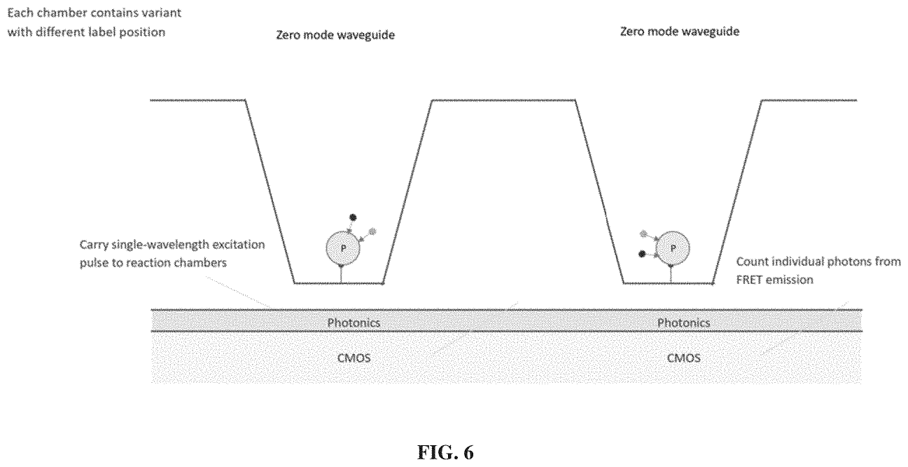

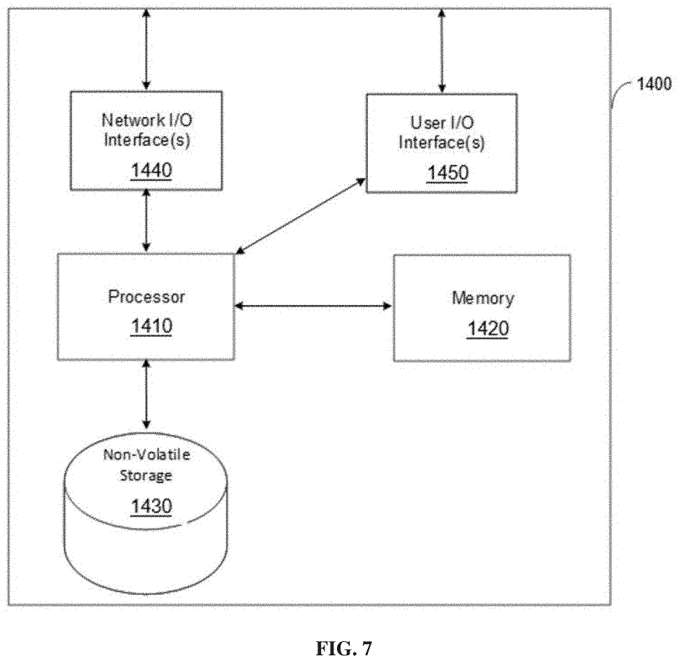

### DETAILED DESCRIPTION

For the majority of proteins, the primary tool for determining protein structure is X-ray crystallography, a tool that has been used to determine crystal structures of proteins since the late 1950s. To date, over 100,000 protein structures were determined at resolution better than 2 angstroms protein structures have been solved using this method. However, X-ray crystallography is time-intensive and expensive (average cost of over $50,000 per protein), is limited to protein structures that are able to form crystals, and provides a static protein structure (i.e., not a dynamic structure, as in solution).

Advances in laser-free electron lasers for hard X-rays, which produce femtosecond X-ray pulses, allows for the structural exploration of ultra-fast events in sub-picosecond time scales. However, the technique is limited to cyclic and reversible reactions triggered by light. The majority of industrial and biomedical applications of proteins involve irreversible reactions such as enzymatically catalyzed reactions. These are typically irreversible, single-pass reactions where substrates bind and are converted into product that is released from the enzyme. Limited dynamic techniques exist to study these reactions but require complex sample mixing techniques in the presence of synchrotron or XFEL x-ray sources. These methods are complex, expensive, and time-intensive to implement.

All crystallography methods are fundamentally limited to protein variants that are able to form crystals at sufficiently high concentrations. Slight variations of the same protein may have completely different crystallization conditions, and many proteins are completely unable to crystalize and are therefore unsuitable for this method.

NMR spectroscopy is also used to obtain high resolution three-dimensional structures of proteins. In contrast to X-ray crystallography, NMR spectroscopy is usually limited to very small proteins (under 35 kDa). It is used to form Conformation Activity Relationships where the structure is compared before and after interaction with a target molecule, such as a drug candidate. The technique is limited due to the crowding and overlapping of the one-dimensional spectrographic signal when larger proteins are analyzed.

Cryogenic electron microscopy (cryo-EM) is another technique for protein structure prediction. Cryo-EM does not require the crystallization of proteins, as aqueous samples of proteins are directly imaged. This greatly increases the number of protein variants that can be imaged with this technology. However, the utility of cryo-EM is currently limited to large proteins and protein complexes due to limitations in resolution. Additionally, cryo-EM is unable to capture time-resolved structures because the sample must be cryogenically frozen, preventing enzymatic activity.

No analytical technology exists to allow for benchtop protein structural determination, either static or dynamic. Such a technology would dramatically increase the speed of protein candidate screening by allowing many candidates to be screened in parallel and in rapid succession with basic laboratory equipment.

Due to the inherent challenges and competing advantages and limitations of the existing methods for empirically elucidating protein structure, there has been a longstanding interest in developing in silico approaches to determining a protein's structure from its amino acid sequence. Many in silico analyses of protein structure and function begin by identifying a protein's “homologs.” Two proteins are considered homologous if they are descended from a common ancestor. Homologous proteins can have substantially different sequences, but they often have similar function and structure. Once a protein of interest's homologs are known, there are several possible in silico routes to protein structure prediction.

In some cases, a 3D structure is not available for the protein of interest, but a 3D structure has already been experimentally gathered for an identified homolog. Since similar amino acid sequences adopt similar structures, an amino acid sequence alignment of the target protein and the homolog as well as the experimentally determined homolog's structure can be used to generate an atomic model of the target protein. This process is called “homology modeling.” If a full-length homologous protein with known structure cannot be found, one can also look for homology between small subsets of the target protein and libraries of shorter homologous sequences, each of which adopt a known fold. This “protein threading” approach can thus be used to build a structure from a collection of short homologous sequences, each contributing a little bit towards defining a portion of the overall structure.

If a protein of interest has no suitable homologous templates, ab initio methods may be used to predict the structure of the protein from amino acid sequences alone. Ab initio methods include physics-based modeling, where thermodynamic and molecular energy parameters are used to propose and rank candidate structures until a minimum entropy/maximum stability model is found.

It is also possible to infer information about a protein's three-dimensional structure by comparing the sequences of homologs and measuring the correlations in amino acid identity at pairs of residues. If two non-neighboring residues are physically in contact, for example by forming a hydrogen bond, then the amino acid identities in these positions will be correlated. Should a mutation at one position occur, it will likely be accompanied by a compensatory mutation in the other residue. In contrast, for two non-neighboring residues that are not in contact, there should be no correlation between their amino acid identities. Co-evolutionary statistical models that capture the tendency of particular pairs of residues to mutate together within a family of protein homologs can thus be used to generate “contact maps” that describe inter residue contacts protein-wide. Contact maps are an important first step towards predicting all inter-residue (pairwise) distances for the amino acids in a protein. Such a distance matrix would be completely descriptive of the 3D structure, and thus, contact maps are an important element of computational protein structure prediction.

### Combination of in Vitro FRET and in Silico Structure Prediction

Fluorescence resonance energy transfer (FRET) can be used to measure the distances between a critical amino acid residue pairs in order to improve (i.e., refine) the performance of a protein structure prediction algorithm by constraining the parameters of the algorithm. For many proteins, a difficulty in running structure prediction algorithms is caused by the existence of many plausible candidate structures that are distinct from the ground-truth structure. These plausible but incorrect candidate structures manifest as spurious local minima in the loss surface of the algorithm. The existence of many spurious local minima significantly increases the difficulty of converging to the correct structure through traditional gradient-based optimization methods. By experimentally determining the physical distances between pairs of amino acid residues of a protein in solution, the inventors of the present disclosure were able to refine a protein structure prediction algorithm in order to produce a superior prediction of individual protein structures.

First, the methods described herein utilize a structure prediction algorithm to identify pairs of amino acids for which distances should be measured (e.g., by determining the estimated distances between all pairs of amino acids using the algorithm and identifying pairs of amino acids based on at least one of several algorithm-predicted factors.

In some embodiments, an algorithm-predicted factor is the degree of variance or uncertainty in the estimated distance between a pair of amino acids. In some embodiments, pairs of amino acids are identified based on identifying pairs that the algorithm estimates have large degrees of variance in their distance measurements. For example, for a given protein sequence, the structure prediction algorithm is first performed to generate an in silico protein structure prediction and a distogram (probability distribution over distances between all pairs of residues). In some embodiments, a pair of amino acids is then identified if the two amino acids are separated on the linear chain by more than approximately five amino acids (i.e., more than five amino acids apart based on primary structure). In some embodiments, the pair of amino acids is identified based on having the distogram element with the highest variance. In some embodiments, the pair of amino acids is identified based on having a distogram element with one of the highest variances (e.g., 2^{nd}, 3^{rd}, 4^{th}, 5^{th}, 6^{th}, 7^{th}, 8^{th}, 9^{th}, or 10^{th} highest variance). Typically, k is between 1 and 100. The variance of a distogram element is a measure of the uncertainty provided by the algorithm about the distance between two amino acids. Selection is limited to only non-neighboring residue pairs because residues that are near each other on the linear chain are trivially close to each other in the physical structure.

In some embodiments, an algorithm-predicted factor is the relative importance of the distance between the two amino acids in the structure prediction algorithm (i.e., how important a particular distance is to the overall predicted structure). The importance of a particular distance relative to another depends on whether it is more or less likely to reduce the global uncertainty for the entire predicted protein structure. There are some distances between pairs of amino acids that are more critical for the algorithm to have as a constraint than others. This can be critical because some peripheral amino acid residues might have high variance or uncertainty in their measurement, but not be important for constraining the algorithm and the ultimately predicted structure. These peripheral amino acid residues might not have many interactions with other residues in the protein. Similarly, some pairs of amino acid residues might have low variance or uncertainty in their distance measurements, but they might be very important for constraining the algorithm and the ultimately predicted structure (e.g., due to their long-range interactions).

In some embodiments, an algorithm-predicted factor is the structural sensitivity of a pair of amino acids. Structural sensitivity may include whether that pair is involved in critical structural support (e.g. salt bridge, disulfide bond, key stabilizing interaction for secondary and/or tertiary structure). If the algorithm ranks a pair of amino acids as a sensitive location because it is critical that they be maintained, the algorithm is likely to de-emphasize the use of this pair for in vitro distance measurements. In contrast, amino acid pairs that that are not structurally sensitive (e.g., in loop regions, not part of a hydrogen bonding network in an alpha helix or beta sheet) would be prioritized by the algorithm for in vitro distance measurements. Structural sensitivity may include whether the amino acid pair is amenable to labeling with a FRET dye. For example, a solvent-exposed single cysteine that is not involved in a disulfide bond or a solvent-exposed lysine are ideal amino acids for labeling and would be ranked highly by the algorithm. In contrast, amino acid residues that would need to be replaced with artificial residues for labeling would be lowly ranked by the algorithm.

Second, the methods described herein involve measuring the distances between identified amino acid pairs in vitro using FRET, inputting those distance measurements into the algorithm to constrain the parameters of the algorithm (e.g., constraining the algorithm's output to agree with the measured distances), and determining, for a second time, a predicted structure of the protein using the refined structure prediction algorithm. From the biophysics of the FRET methodology, there will be an estimate for the uncertainty in the distance measurement. The distogram output of the algorithm can be constrained such that the averages of the amino acid pair distances are the empirically FRET-measured values and the uncertainty of the amino acid pair distances are the standard deviations of the FRET-measured values. In some embodiments, this constraining of the algorithm is performed by setting the distributions of the FRET-measured values to be Gaussian with mean and standard deviation set as described above. With this new distogram, which is constrained to match the FRET-measured distances, the protein structure prediction algorithm may be run again to generate a more accurate and refined protein structure, starting with the distograms and angleograms.

### Direct Coupling Analysis

When generating contact map predictions, it is necessary to go beyond the raw correlations, due to the fact that some observed correlations may indirect. For example, if residue A interacts with residue B, and residue B interacts with residue C, there will be a substantial correlation between residues A and C, but no true contact between A and C. To leverage co-evolutionary data for accurate structural determination, it is necessary to distinguish direct and indirect correlations. The state-of-the-art algorithm for deducing direct correlations is called Direct Coupling Analysis (DCA). Once a collection of all the known protein sequences that are homologous to a protein of interest have been assembled into a multiple sequence alignment (MSA), direct coupling analysis (DCA) can be performed to solve a Potts model on the alignment. The output of (DCA) is a matrix that represents the “strength” of the coupling between all pairs of residues. Empirically, it has been demonstrated that a high DCA output value often indicates that the two residues are physically in contact. The quality of the DCA analysis is measured by the extent to which the output, when threshold appropriately, produces accurate predictions for whether or not each pair of residues is in contact (defined by being within a certain distance from each other). Using a predicted three-dimensional structure based on DCA, one can identify pairs of amino acids that have high variance in the spatial distance between the two amino acids. As described herein, researchers may then take these amino acids identified in silico and determine the experimental distance between them in vitro, e.g., in order to refine the DCA predictions and/or the protein structure prediction models.

Three-Dimensional Structure Prediction from DCA Generated Contact-Maps

Computer-implemented protein structure prediction models (e.g., neural network models) may be applied to predict the three-dimensional structure of the protein from the contact maps generated by DCA. In some embodiments, a protein structure prediction model is AlphaFold, as developed by Google DeepMind. In some embodiments, a protein structure prediction model is any prediction model that is currently known or developed in the future. In some embodiments, a protein structure prediction model is a refinement of a previously described protein structure prediction model (e.g., a refinement of AlphaFold).

In some embodiments, a protein structure prediction model comprises four primary steps:

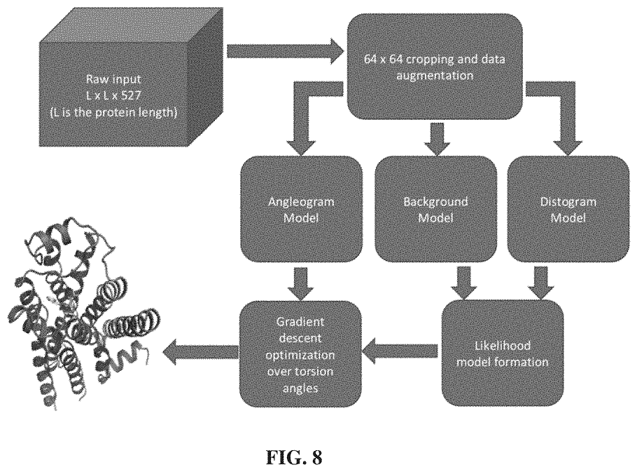

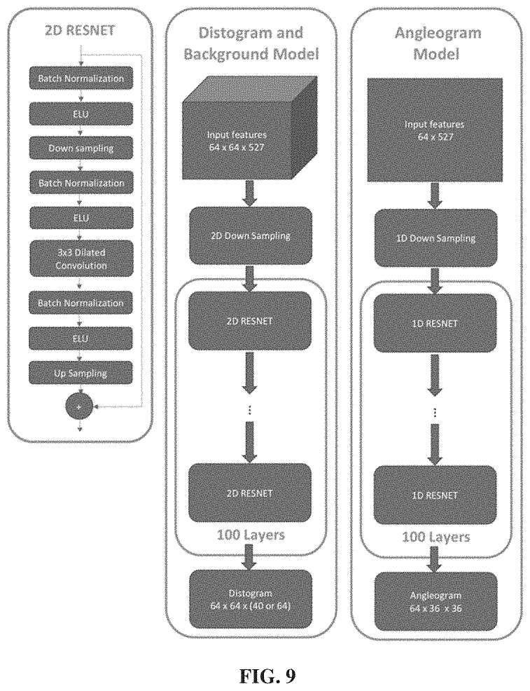

(1) Posterior distribution estimation. These estimations are trained with full knowledge of the statistical features and amino acids of a target protein (shown as “distogram model” in FIG. 8). In some embodiments, the posterior estimator is a 2D Resnet, optionally with 220 layers, which is trained with a full set of input information (FIG. 9).

(2) Prior distribution estimation. These estimations are based on protein length and locations of Glycine amino acids (shown as “background model” in FIG. 8). The prior distribution estimation entails a similarly structured Resnet as the posterior distribution estimation but is trained on different input. (FIG. 9).

(3) Torsion angles distribution estimation. These estimations are used as initialization generative model in maximum likelihood (ML) estimation of protein structure (shown as “angleogram model” in FIG. 8). In some embodiments, the angleogram distribution estimator is a 1D Resnet which has a structure similar to the posterior estimations. The input is also similar to the inputs for the posterior estimations, but the output is the joint distribution over (Φ, Ψ, Ω) torsion angles. The initial angle estimation is important for the optimization process as the final folding model is highly dependent on it.

(4) Solving a maximum likelihood estimation by optimizing over two torsion angles. To perform maximum likelihood estimation over each protein structure (e.g., the distance matrix), a differentiable model from torsion angles to distance matrix is required. To reduce the complexity of this problem, it is assumed that the C—C and C—N bound lengths are fixed to a predefined value and the torsion angle is fixed to 180 degrees. In some embodiments, this step is implemented using Torch or Tensorflow. These functions are flexible to incorporate all bond lengths and torsion angles to the optimization process.

A protein structure prediction model may be implemented for protein structure prediction downstream of DCA-based feature extraction. In some embodiments, prior, posterior and angleogram models may be trained by applying random croppings of full pairwise features. These crops are designed to cover the full protein but with random onsets. This leads to a data augmentation process that prevents the model from over fitting and makes it robust to shifts in the peptide chain. To predict the 3-D structure of each protein, protein feature extraction is first performed by computing Potts model parameter and applying DCA. The prior and posterior distograms are then obtained using these features. The likelihood function is then obtained by dividing the posterior estimations over the prior estimations. The final step of optimization is to perform a repeated gradient descent over the (Φ, Ψ, Ω) torsion angles.

### Generative Model Provide Good Structure Initializations

The maximum likelihood (ML) optimization surface is non-convex and will include many local minima and saddle points. To mitigate that issue, one may start the gradient descent from model-guided initial presumptions. Model-guided initial presumptions can be obtained by sampling a target protein's angleogram multiple times and/or by generating many samples using a variational encoder-decoder; and then computing a distance matrix for each initialization point. From this selection of initialization points, one can select the points with the highest structural scores.

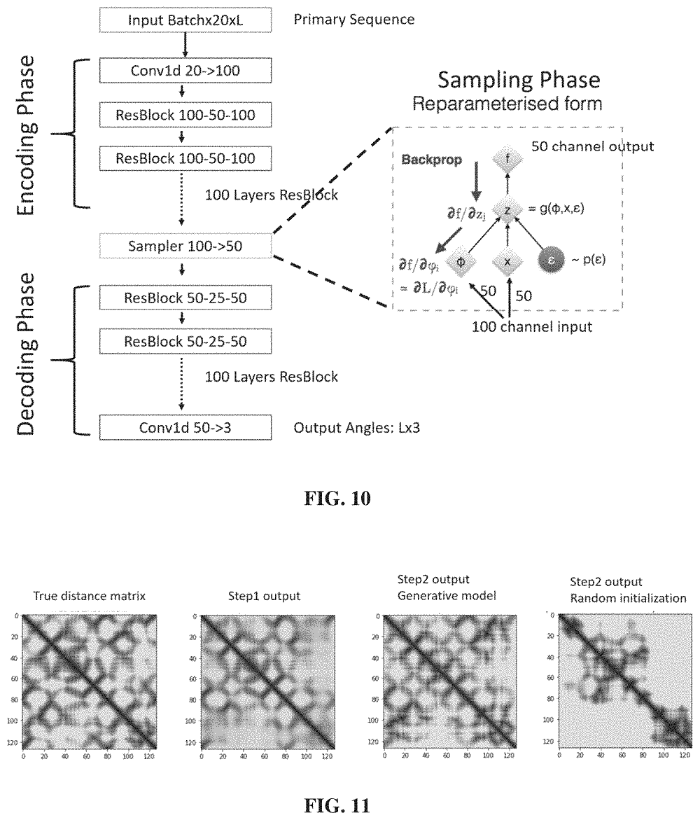

In order to obtain a good starting population of candidate protein structures, the inventors have developed a 1D deep resnet generative model (FIG. 10) from the primary sequence to protein structure, wherein each structure is represented by a sequence of triplet dihedral angles (Φ, Ψ, Ω). This generative model is designed to sample different possible structures, such that many candidate structures can be obtained from a single primary sequence. Initializing gradient descent with many candidate structures from a generative model improves the final model output, which is a distance matrix capturing the structure of the target protein, relative to random initialization (FIG. 11).

The 3-D backbone structure of a target protein could be represented by cartesian coordinates of protein backbone atoms (alpha-carbon, beta-carbon and N terminal) or by a list of torsion angles of the protein backbone structure. Because cartesian coordinates of protein backbone atoms can be directly converted to a sequence of triplet dihedral angles (Φ, Ψ, Ω), a “sequence to structure” model takes the primary sequence input as a list of one-hot vector(s) (20 dimension) and output structure(s) as a list of torsion angles. For a protein structure with L amino acid residues (L×20 matrix), the structure could be represented by a L×3 matrix (i.e., 3 torsion angles (Φ, Ψ, Ω)). This model, which comprises three discrete phases, is described in FIG. 10 and below:

(1) Encoding phase. The input layer is propagated through the Conv1D project (20 dimension to 100 dimensions), which generates a 100×L matrix. This matrix is iterated 100 times through a residual network (RESNET) block (FIG.ResBlocklD) that performs batch norming, applies the exponential linear unit (ELU) activation function, projects down to 50×L, applies again batch norming and ELU, and then cycles through 4 different dilation filters. The dilation filters have sizes 1, 2, 4, and 8 that are applied with a padding of the same to retain dimensionality. The final batch norm, the matrix is projected up to 100×L and an identity addition is performed.

(2) Sampling phase. A 100×L matrix is generated from the encoding phase, and the first 50 dimensions from the encode vector in each position serve as the mean of 50 gaussian distributions and last 50 dimensions serve as the log of variance of the corresponding gaussian distributions. After applying a reparameterization trick, the model samples the hidden variable z from 50 gaussian distribution, which together generates 50xL matrix as output.

(3) Decoding phase. The input for the decoding phase is the 50xL matrix output from the sampling phase, and it iterates a similar ResBlock as in the encoding phase for 100 times (The primary difference from the encoding phase ResBlock is that the ResBlock module of the decoding phase maps 50 dim to 50 dim input). After ResBlock layers, the model reshapes the 50 dimension to 3 dimension (corresponding to 3 torsion angles) using 1D convolution with kernel size 1.

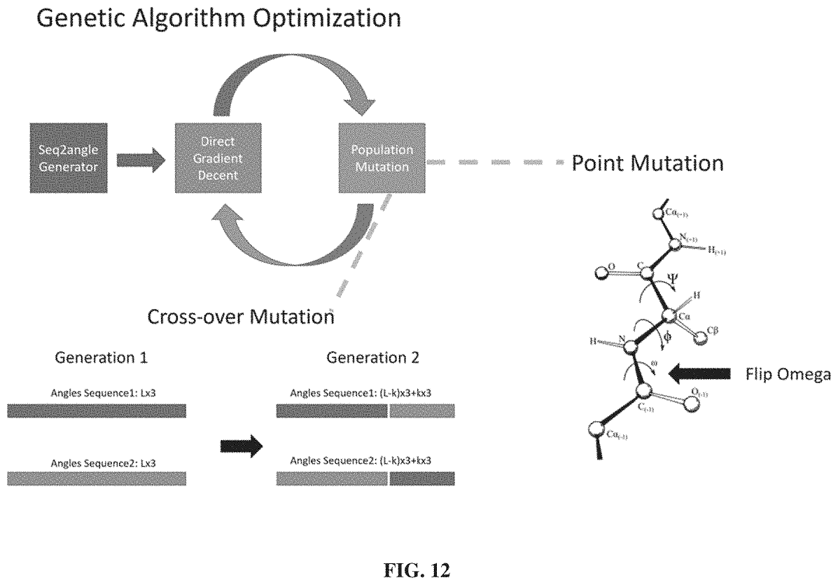

The initial starting point is important for gradient descent optimization. After experimenting with different global optimization approaches, it was found that a genetic algorithm (GA) with two specific mutation operation works well for structure prediction (FIG. 12)

Given a primary sequence, the generative model described above may be used to generate 200 candidate structures as an initial population. Each structure may be represented by a sequence of triplet dihedral angles (Φ, ψ, Ω). Direct gradient-descent optimization for each structure in the 200 may be implemented. After at least 1,000 direct gradient-descent steps, the genetic algorithm (cross-over mutation within 200 population and randomly select position to flip the Omega angle) may be used as a new generation for direct optimization. After each round of GA interaction, one may keep the highest performer (without cross-over) in the new population.

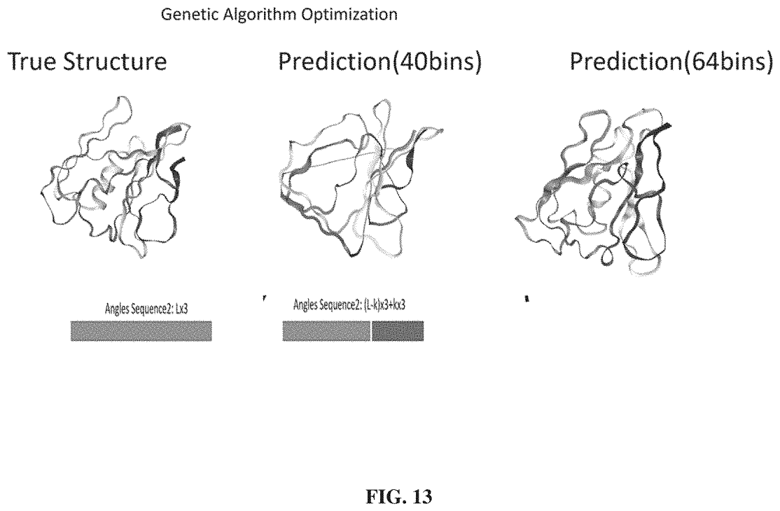

The inventors of the present disclosure have found that a protein structure prediction model such as AlphaFold, with 40 bins could learn a high-performing pair-wise distance matrix. In some embodiments, the step1 model may be re-trained to output 64 bins to cover distance range 0 Å to 32 Å (0.5 Å per bin). The 64-bin framework gives high resolution and reveals better local structure detail. See FIG. 13.

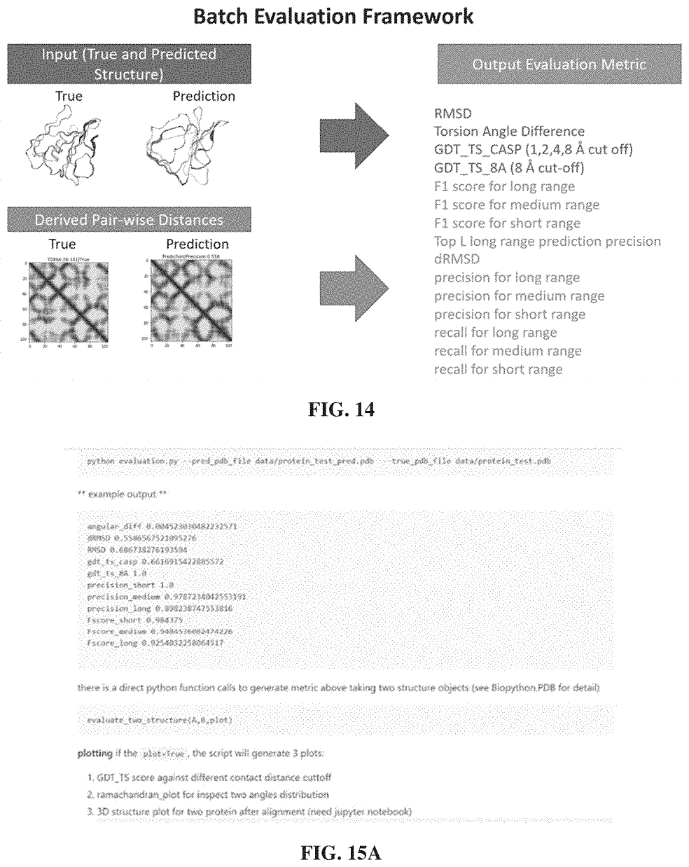

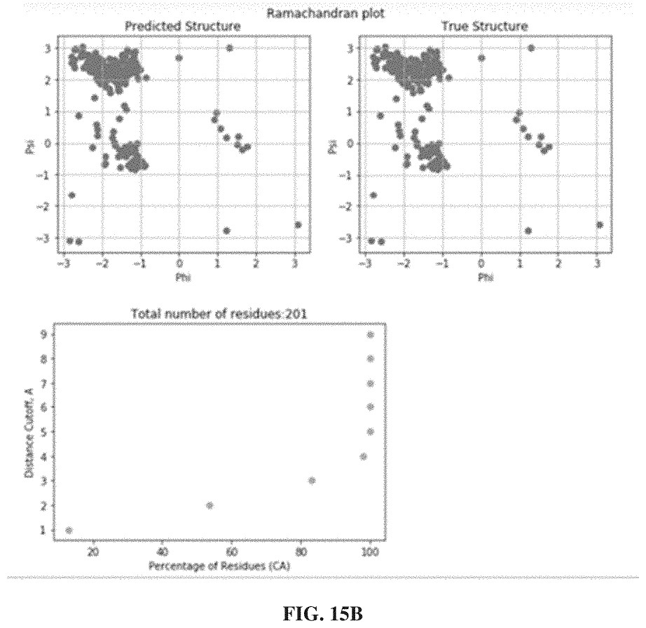

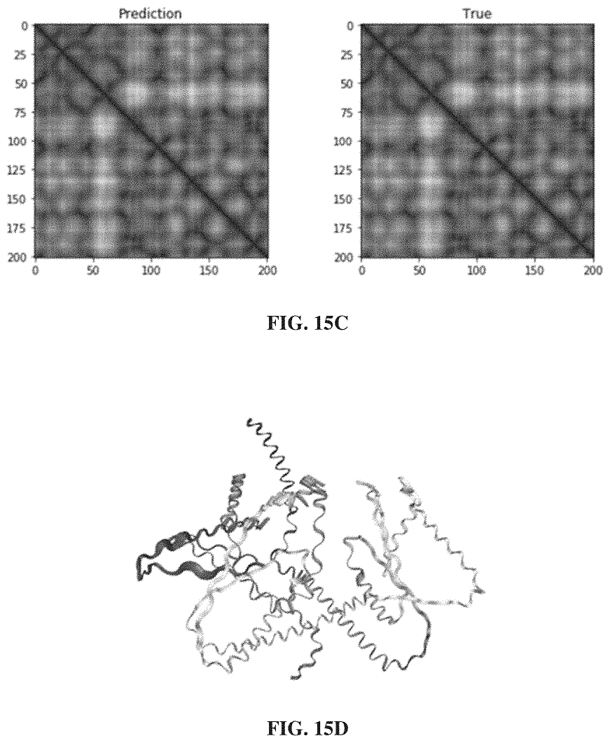

A set of evaluation/convert/plotting python scripts have been developed to allow for acquisition of a unique metric used (dissimilar from previously reported metrics) for ascertaining how well a model algorithm predicted a given protein's structure (FIG. 14). The evaluation framework also contains built-in visualization. (FIG. 15).

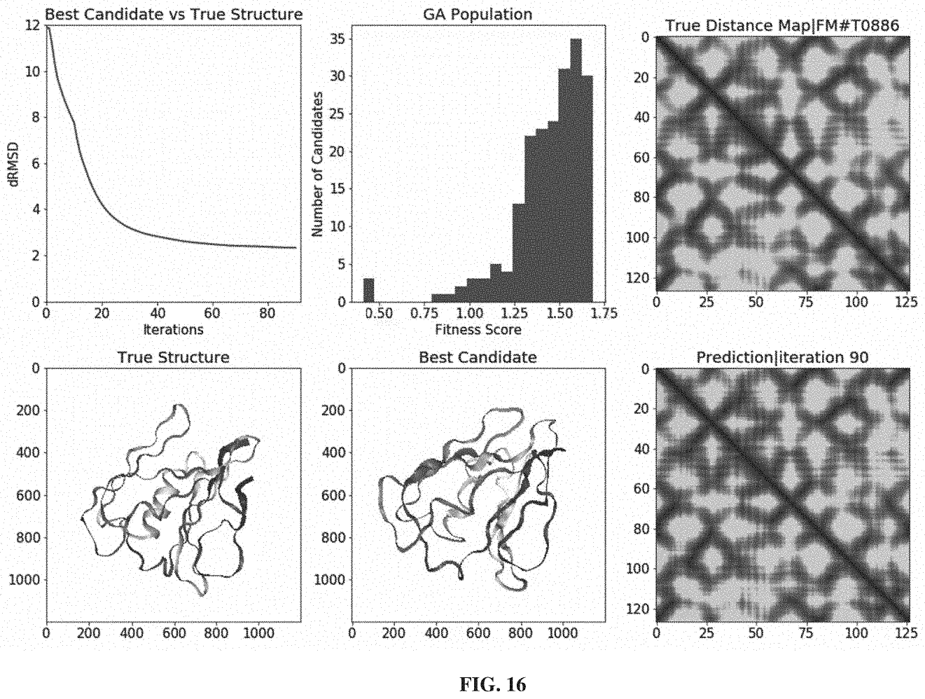

In some embodiments, a fully implemented in silico protein sequence to structure prediction has been performed. An example predicted structure versus the ground-truth structure is shown in FIG. 16.

### Identification of Sites for Measurement

Identifying a pair of two amino acids that should be labeled for determination of the distance between them can be a challenging problem for several reasons. First, for an average protein comprising a length of 500 amino acids, empirically measuring the distance between every pair of amino acids in vitro would be impractical (protein of 500 amino acids has ˜125,000 pairs of amino acids). Second, many of the amino acids of a given protein (e.g., glycine residues) are not amenable to labeling with fluorescent dyes, and swapping these amino acids for ones that could be labeled would have a high probability of destabilizing the protein structure. Therefore, care must be taken to pick residues that are least likely to disrupt the protein structure and that will maximally improve the accuracy and usefulness of the structure model of the protein of interest. Furthermore, a maximum of two available labeling sites should be chosen for each protein variant, ideally wherein each amino acid site for labeling is an estimated 2-10 nanometers from one another. In some embodiments, the two amino acids in a pair of amino acid residues in a protein are estimated to be about 2, 3, 4, 5, 6, 7, 8, 9, 10, 11, 12, 13, 14, 15, 16, 17, 18, 19, or 20 nanometers apart from one another.

In some embodiment, labeling is done at two solvent-accessible cysteines or lysines or a combination of the two that are within 10 nanometers but may or may not be forming disulfide bonds with each other. In one embodiment, all of the native cysteines but one or two are replaced with other amino acids that cannot be labeled. Cysteines that form disulfide bonds with other cysteine may not be necessary to get rid of as they are likely locked into their disulfide bonds and serve an important stabilizing function for the protein structure and furthermore may be nonreactive with FRET dyes.

In some embodiment, the two amino acids of a pair are solvent-exposed (or solvent-accessible). In some embodiments, at least one of the two amino acids of a pair is a solvent-exposed essential amino acid. In some embodiments, at least one of the two amino acids of a pair is a naturally-occurring amino acid. In some embodiments, at least one of the two amino acids is a cysteine or lysine. In some embodiments, at least one of the two amino acids of a pair is a wild-type amino acid of the protein. In some embodiments, at least one of the two amino acids of a pair has been mutated from its wild-type amino acid. In some embodiments, at least one of the two amino acids of a pair is a non-natural amino acid. In some embodiments, a non-natural amino acid is mutated into the protein. In some embodiments, the non-natural amino acid is p-azido-L-phenylalanine (AZF) (e.g., replacing a native/wild-type phenylalanine). Examples of non-natural amino acids that can be used for site-specific protein labeling may include 1: 3-(6-acetylnaphthalen-2-ylamino)-2-aminopropanoic acid (Anap), 2: (S)-1-carboxy-3-(7-hydroxy-2-oxo-2H-chromen-4-yl)propan-1-aminium (CouAA), 3: 3-(5-(dimethylamino)naphthalene-1-sulfonamide) propanoic acid (Dansylalanine), 4: Nε-p-azidobenzyloxycarbonyl lysine (PABK), 5: Propargyl-L-lysine (PrK), 6: Nε-(1-methylcycloprop-2-enecarboxamido) lysine (CpK), 7: Nε-acryllysine (AcrK), 8: Nε-(cyclooct-2-yn-1-yloxy)carbonyl)L-lysine (CoK), 9: bicyclo[6.1.0]non-4-yn-9-ylmethanol lysine (BCNK), 10: trans-cyclooct-2-ene lysine (2′-TCOK), 11: trans-cyclooct-4-ene lysine (4′-TCOK), 12: dioxo-TCO lysine (DOTCOK), 13: 3-(2-cyclobutene-1-yl)propanoic acid (CbK), 14: Nε-5-norbornene-2-yloxycarbonyl-L-lysine (NBOK), 15: cyclooctyne lysine (SCOK), 16: 5-norbornen-2-ol tyrosine (NOR), 17: cyclooct-2-ynol tyrosine (COY), 18: (E)-2-(cyclooct-4-en-1-yloxyl)ethanol tyrosine (DS1/2), 19: azidohomoalanine (AHA), 20: homopropargylglycine (HPG), 21: azidonorleucine (ANL), 22: Nε-2-azideoethyloxycarbonyl-L-lysine (NEAK).

In some embodiments, at least one of the two amino acids of a pair is labeled using an N-terminal transglutaminase. In some embodiments, labeling is done between N-terminal transglutaminase and a non-natural amino acid with orthogonal chemistry (such as functional p-azido-L-phenylalanine (AZF) group).

In some embodiments, the pair or pairs of amino acids are chosen at random to replace with a non-standard amino acid (e.g. AZF). In some embodiments, all solvent-exposed native cysteines and/or lysines are labeled with FRET dyes.

In some embodiments, a researcher uses a protein structure prediction model (e.g., a coarse protein structure prediction model) to identify amino acid residues that are amenable to labeling with a FRET dye molecule. In some embodiments, a researcher uses a protein structure prediction model (e.g., a coarse protein structure prediction model) to identify amino acid residues that are amenable for mutation to introduce an amino acid (e.g., cysteine, lysine, or a non-natural amino acid) that can be labeled with a FRET dye. Amino acid residues that are amenable for labeling or mutation can be labeled or mutated without significant disruption to the conformation of the protein (e.g., are solvent-exposed, in an active site or located outside of a structural domain). In some embodiments, the protein structure prediction model is a protein folding algorithm. In some embodiments, the protein structure prediction model identifies at least one pair of amino acids on the surface of the protein for which the model cannot predict their locations (e.g., distances from one another) with a high degree of accuracy and/or precision. In some embodiments, the protein structure prediction model identifies at least one pair of amino acids that would benefit from increased resolution of their location (e.g., location of one amino acid of the pair relative to the other). In these embodiments, the protein structure prediction model first predicts the relative locations of all of the amino acids on the surface of the protein relative to one another in order to produce a distogram or distance matrix.

Once all the surface residues of the protein are identified, a single residue may be chosen for the first label. In some embodiments, this single residue is a cysteine that is not a part of a disulfide bond or a lysine. The algorithm may predict whether the single residue is an element of a stabilizing force of the protein (e.g., element of a disulfide bond). If the single residue is mutated, the algorithm will provide a listing of optional amino acids for mutation that are chemically similar to the native amino acid in order to not disrupt the conformation or stability of the protein. Then, the algorithm may draw a sphere and identify all other cysteines, lysines, or replaceable amino acids within a 10 angstrom radius. If the algorithm locates any other of these amino acids, it may again check to see whether this is a solvent-accessible amino acid. If it is, this may be chosen to be the second amino acid of the pair for labeling.

In some embodiments, in order to identify surface exposed residues, the protein structure prediction model first checks for protein loops. The protein structure prediction model may then check for possible disruption of secondary structure, and then locate all potential pairs of amino acids that can be labeled or mutated.

In some embodiments, the protein structure prediction model (e.g., protein folding algorithm) further refines the selection of a pair of amino acid by suggesting amino acid residues that maximally collapse the number of possible solution sets. In some embodiments, the algorithm determines the estimated distance between each and every possible solvent-exposed amino acid residue. In some embodiments, the algorithm then produces a distogram (or matrix of distances between each possible pair of amino acids) and rank orders each possible pairing of amino acids based on one of several factors (e.g., the uncertainty or variance in the measurement of the distance between each pairing). The algorithm may then use this ordered list of possible amino acid pairs (e.g., ranked from highest uncertainty or variance to least uncertainty or variance) to identify at least one pair of amino acids that could be labeled with a FRET dye or mutated to allow for labeling with a FRET dye. In vitro experimental determination of the distance between the two identified amino acid residues can then be used to refine the algorithm by constraining the possible distance between the pair of amino acids during subsequent predictions of the structure of the protein.

### Methods for Labeling

For a given protein, pairs of amino acids on the surface of the protein are chosen to be labeled by FRET dyes. In some embodiments, the pairs of amino acids are amenable to labeling (e.g., cysteine, lysine). In other embodiments, one or both of the amino acids of a pair is a native amino acid that is not amenable to labeling (e.g., glycine). Amino acids that are not amenable to labeling can be mutated to natural amino acids that are amenable to labeling (e.g., cysteine, lysine) or to non-natural amino acids having functional chemical groups that are amenable to labeling.

In some embodiments, amino acids are labeled with FRET dye molecules. One amino acid of a pair can be labeled with a FRET donor molecule and the second amino acid of the pair can be labeled with a FRET acceptor molecule. FRET pairs are typically chosen at an estimated distance between one and ten nanometers, and when possible (based on limited computational structure predictions) amino acid pairs should be chosen in this range for maximum accuracy. FRET dyes are typically decorated near the active site of the protein, in an inert area, or on the N or C terminus of the protein.

In some embodiments, a FRET molecule is a small organic dye, a fluorescent protein, or a quantum dot. In some embodiments, a fluorescent protein for use in FRET is as described in Bajar, B. T., “A Guide to Fluorescent Protein FRET Pairs” Sensors (Basel). 2016 September; 16(9): 1488.; the entire contents of which are incorporated herein by reference. In some embodiments, a FRET pair (i.e., FRET donor and FRET acceptor) is selected from cyan fluorescent proteins (CFPs) and yellow fluorescent proteins (YFPs), green fluorescent proteins (GFPs) and red fluorescent proteins (RFPs), far-red fluorescent proteins (FFPs) and infrared fluorescent proteins (IFPs), large Stokes shift fluorescent proteins (LSS FPs) and fluorescent protein acceptors, dark fluorescent proteins, and phototransformable fluorescent proteins. In some embodiments, an organic dye typically comprises aromatic groups, planar or cyclic molecules with several π bonds. Exemplary dyes include Alexa Fluor 488 (AF488), Alexa Fluor 647 (AF647), and Texas Red. Additional fluorophores utilized in some embodiments of the methods described include fluorescein, rhodamine, coumarin, cyanine, Oregon Green, other Alexa Fluor dyes besides AF488 and AF647, eosin, dansyl, prodan, anthracenes, anthtraquinones, cascade blue, Nile Red, Nile Blue, cresyl violet, acridine orange, acridine yellow, crystal violet, malachite green, BODIPY, Atto, Tracy, Sulfo Cy dyes, HiLyte Fluor, and derivatives of each thereof. Further non-limiting examples of useful dyes are known in the art (see, e.g. Stockert, J. C and Blázquez-Castro, A. Chapter 3 Dyes and Fluorochromes, Fluorescence Microscopy in Life Sciences. 2017, Bentham Science Publishers. pp. 61-95.; Herman B. Absorption and emission maxima for common fluorophores, Curr. Protoc. Cell Biol. 2001, Appendix 1:Appendix 1E.).

To conjugate a FRET pair onto a protein's surface, several site-specific labeling techniques may be used. These techniques may be used independently of one another or in combination. The most important factor is that only two FRET dyes are conjugated to the protein, and that the dyes are applied to surface residues so as not to disturb or unfold the protein and generate a false signal.

FRET pairs are placed on the surface of the protein using either a combination of natural and unnatural (or non-canonical) amino acids, or exclusively unnatural amino acids. Methods for decorating cysteine residues with fluorescent dyes are widely published. In some embodiments, two canonical amino acids such as cysteines or lysines, ideally on the surface of the protein, are labeled with two separate FRET dyes. For maximum control of this labeling, all native cysteines are replaced with other non-reactive amino acids such as alanine or serine so that cysteines may be introduced at specific sites in the protein. Ideally the native amino acids at these sites are similar in chemical composition to cysteine so that when they are replaced by cysteine, the protein's structure is not disturbed.

The most common way to achieve site-specific labeling is to conjugate the thiol group of cysteine and the amino group of lysine amino acid (AA) residues present in proteins with commercially available maleimide and succinimide dyes, respectively (Stephanopoulos and Francis, 2011). Labeling through cysteines is more attractive for site-specificity because of the low abundance of cysteines in most protein sequences (cysteines are the second most rare of all 20 AA). Clearly, this strategy has limitations for proteins where cysteines are critical for folding and function of the protein or where more than two native cysteines already exist in the protein chain.

Cysteines are preferred because they are less frequent in natural proteins. They are the second rarest amino acid. Lysines are still doable but less preferred because they are very frequent in natural proteins. Amine-reactive conjugates, such as succinimidyl-esters or isothiocyanates, can be used to label lysine residues or N-terminal amines. Care must be taken to not disrupt stabilizing bonds such as disulfide bonds.

An even mix of two FRET dyes is conjugated onto the two exposed cysteines for a maximum theoretical labeling efficiency of 50% (50% will have correct pairing of Donor and Acceptor dyes, i.e. AD, DA, while 25% will have AA and 25% will have DD).

In some embodiments, non-canonical amino acids are introduced to the protein. These amino acids are chosen to be bioorthogonal such that a FRET pair may be selectively conjugated onto the non-canonical amino acid, by way of a reaction such as click chemistry, but are not conjugated onto any natural amino acid. It is important the non-canonical amino acids to not overly disturb the local or global protein structure as this would defeat the purpose of precise distance measurements. Propargyllysine and p-acetylphenylalanine(AcF) are examples of unnatural amino acids. Propargyllysine is an unnatural amino acid which, when incorporated into a protein, can be exploited to attach commercially available fluorescent azide dyes through copper-catalyzed alkyne-azide cycloaddition click reaction (also known as click reaction). p-acetylphenylalanine (AcF), whose ketone functional group can be ligated with hydroxylamine dyes (Brustad et al., 2008). This reaction is optimally carried out at low pH, which makes it less attractive for some biological applications.

Single non-canonical amino acids are introduced at pairs of sites. They are encoded by recoded rarest stop codons, or by an expanded genetic alphabet. Labels are added with 50% theoretical efficiency, which is the same as cysteine labeling. Two non-canonical amino acids are introduced with orthogonal click chemistries. They are encoded by two rarest recoded stop codons, or by an expanded genetic alphabet. Labels are added with 100% theoretical efficiency and they are a combination of canonical and non-canonical amino acids.

### Measurement

Fluorescence energy transfer is understood as the transfer of energy from a donor dye to an acceptor dye during which the donor emits the smallest possible amount of measurable fluorescent energy. A fluorescent dye donor is for example excited with light of a suitable wavelength. Due to its spatial vicinity to an acceptor, this results in a non-radiative energy transfer to the acceptor. When the second dye is a fluorescent molecule, the light emitted by this molecule at a particular wavelength can be used for quantitative measurements. In some embodiments, the donor is excited and converted by absorption of a photon from a ground state into an excited state. If the excited donor molecule is close enough to a suitable acceptor molecule, the excited state can be transferred from the donor to the acceptor. This energy transfer results in a decrease in the fluorescence or luminescence of the donor and, if the acceptor is luminescent, results in an increased luminescence. The efficiency of the energy transfer depends on the distance between the donor and the acceptor molecule. The decrease in signal depends on the separation distance.

In some embodiment, FRET measurements are taken in bulk in a microtiter plate. In some embodiments, a single well in a microtiter plate contains millions of copies of the same protein and FRET-labeled amino acids. FRET measurements may be collected using an apparatus such as a plate reader to measure bulk fluorescence intensity. FRET-labeled pairs will vary from well to well.

The fluorescence intensity can be measured on any device capable of measuring fluorescence either in bulk or with single molecule resolution to determine the distance between these amino acids. Standard FRET measurement techniques are used to determine distances based on FRET intensity from either the fluorescence intensity or fluorescence lifetime. In some embodiments, a positive control (e.g., a FRET-labeled peptide having a known distance between the FRET pair) can be used to assist in defining the transfer function between FRET intensity and distance measurement.

In some embodiments, measurements are taken using FLIM (fluorescence lifetime imaging). The fluorescence lifetime of the donor fluorophore is reduced during energy transfer, a process that can be imaged using FLIM. FLIM builds an image based around differences in the exponential decay of fluorescence (i.e., fluorescence lifetime). This method is particularly useful because it can discriminate fluorescent intensity changes due to the local environment and it is insensitive to the concentration of the fluorophores.

In some embodiments, FRET measurements are taken using fluorescence anisotropy. Anisotropy measurements are based upon the rotation (rotation correlation time) of a fluorescent species within its fluorescence lifetime, described in detail. Two parameters are crucial for these measurements: the fluorescence lifetime and the size of the label. If the lifetime is too short, the population will appear highly anisotropic, whereas, if it is too long, the species will have low anisotropy. Fluorescein with a lifetime of 4 ns is useful for this application. Anisotropy measurements are particularly suited when one protein is significantly smaller than the other. When binding to the larger protein, the anisotropy of the smaller unit increases because the larger complex has a slower rotation correlation time. This provides a sensitive measurement of complex formation. However, when a large label is used, as for instance a fluorescent protein, then the rotation is inherently slow giving rise to high anisotropy values, which compromises the sensitivity of the measurements. Therefore, they should be avoided.

In some embodiments, the measurements are taken at the single molecule level in an apparatus such as a zero-mode waveguide. A zero-mode waveguide comprises discrete chambers (or wells), wherein each chamber contains a separate copy of the protein with a different FRET pair. In a zero-mode waveguide based apparatus, each protein variant with its unique label pair resides in its own chamber, and therefore, each chamber measures an independent distance measurement.

In some embodiments, the protein of interest is attached to the surface via a biotin-streptavidin link. The bottom surface of the zero mode waveguide is functionalized with a biotin tethered to a high-density PEG coating. The biotin is attached to a streptavidin intermediary, which then binds to another biotin on the surface of the protein of interest. The final attachment order is: ZMW Surface: PEG-biotin: Streptavidin: biotin-protein. A maximum of one streptavidin-bound protein must sit in each zero mode waveguide to avoid overlapping signal.

In some embodiment, the FRET pairs are measured using a conventional fluorescence microscope. In some embodiment, the FRET pairs are measured using a total internal reflection fluorescence (TIRF) microscope.

In some embodiment, FRET measurements are obtained using a dynamic structure of the protein interacting with a substrate. This would require a single molecule imaging device with time-series data collection, such as a zero mode waveguide or TIRF microscope. Once the protein variants have been bound to the imaging surface, reaction substrate can be injected at high concentration to catalyze a protein reaction or initiate a protein-substrate binding event. Because each molecule is imaged independently, the distance change in each FRET pair can be aligned via software after the measurement point. This provides a large advantage over dynamic X-ray crystallography, which requires that each protein must react with the substrate at the exact same time in order to be imaged as a single synchronized crystal. This means that a much wider variety of reaction types can be assayed beyond light-activated reversible reactions. In some embodiments, these methods enable measurement of distances involved in non-reversible reactions.

In some embodiments, the total measurement time last for 30 seconds due to inevitable photo-bleaching from the laser excitation. In some embodiments, the total measurement time lasts for 1-60, 5-60, 10-60, 20-60, or 30-60 seconds. This provides sufficient time to collect measurements to construct both the static and dynamic crystal structures. This also provides enough time to flow in a ligand of interest or otherwise change the buffer conditions to see how the protein being assayed changes conformation

### Barcoding

In some embodiments, for imaging methods where physical segregation is used to separate variants (e.g., imaging in a microtiter plate or zero-mode waveguide), the individual protein variants do not need to be barcoded (e.g., with a unique molecular identifier). In some embodiments, for imaging methods where physical segregation is used to separate variants (e.g., imaging in a microtiter plate or zero-mode waveguide), the individual protein variants are barcoded.

In some embodiments, for methods to identify which two amino acids have been labeled after the single-molecule FRET measurements have been taken, the proteins are barcoded. Barcoding of a protein variant can be done in any conceivable way known to a person of skill in the art (e.g., polypeptide sequencing).

In some embodiments, the barcode of a protein variant comprises a short, protein-bound, nucleic acid-based unique molecular identifier. In some embodiments, the barcode of a protein variant comprises a complete protein-coding nucleic acid sequence. In some embodiments, the barcode of a protein variant is its amino acid sequence.

An in vitro genotype-phenotype link can be established in several ways, including via ribosome display, direct RNA binding, mRNA display, phage display, yeast display, or via the construction of a fusion protein with a DNA-binding domain.

Depending on the type of barcode used, various readout methods may be employed. If a random nucleic acid sequence barcode is used, complementary fluorescently labeled DNA, RNA, LNA, or PNA probes can be introduced to the bulk sample at high concentration and hybridized to the unique barcodes. In order to create a great enough number of protein variants, combinations of fluorophores can be used to create unique visible signatures. This will likely limit the number of detectable protein variants to double-digits.

If a direct genotype-phenotype link is created, nucleic acid sequencing on a zero mode waveguide sensor allows for the most accurate identification of a high number of variants (thousands to millions). If ribosome display was used to link the coding RNA to the protein of interest, a reverse transcriptase reaction coupled with single-molecule DNA sequencing on a PacBio system can be employed to recover the coding DNA sequence. If a fusion DNA-binding protein is formed, direct single-molecule DNA sequencing on a PacBio system may be used to recover the DNA sequence. If no genotype-phenotype link is created, single molecule peptide sequencing may be used to identify individual amino acid residues.

### Refining the Algorithm

In some embodiments, after FRET-determined distance measurements are collected for multiple pairs of amino acids in a protein, these measurements are used to refine a distogram, wherein each entry in the matrix is a probability distribution that captures the likelihood of the distance from one amino acid to every other amino acid. In some embodiments, the most effective use of the FRET-based distance measurements is in conjunction with a computational protein folding prediction model. In some embodiments, the distogram is a component of protein folding prediction algorithms. The distogram may be combined with predicted angles between the amino acid backbone and predicted distances (e.g., with statistical uncertainty or a distogram) between each amino acid to recover a complete protein structure. The distances generated by FRET measurements, in some embodiments, act as constraints on a structure prediction algorithm (e.g., a computational protein folding model). In some embodiments, constraining the algorithm decreases the total computational time to determine the structure of a protein (e.g., by at least 10%, 20%, 30%, 40%, 50%, 75%, or 100%). In some embodiments, constraining the algorithm leads to a more accurate prediction of the structure of a protein of interest.

In some embodiments, an algorithm is a probabilistic model that generates a posterior angelogram and a distogram (e.g., a probabilistic matrix of the angles and distances, respectively, between every amino acid).

In some embodiments, the algorithm will find multiple solutions that minimize the energy landscape described by the distogram. However, once the FRET labeling provides the ground-truth distances between several locations, solution structures of a protein can be eliminated that diverge (i.e., fall outside of a specified range) from the distances measured by FRET between the amino acid residues.

In some embodiments, it is envisioned that the algorithm will be implemented by a computer processor.

### Computer Implementation

Some aspects of the present disclosure provide a computer-implemented method comprising at least some of the following steps: performing in silico a three-dimensional structure prediction of a protein using a structure prediction algorithm; identifying in silico at least one pair of solvent-exposed amino acids in the protein based on algorithm-predicted factors (e.g., variance in the spatial distance between the two amino acids of the at least one pair); and constraining the structure prediction algorithm using distance measurements collected in vitro between amino acids of the at least one pair of amino acids present in a recombinant copy of the protein using fluorescence resonance energy transfer (FRET), wherein a FRET donor is attached to one amino acid of the pair and a FRET acceptor is attached to the other amino acid of the pair.

In such an implementation, it is envisioned that software is written in any suitable programming language such that when implemented by a processor causes that processor to perform the steps of the method. The software may have artificial intelligence machine learning algorithms, trained by an initial set of training data, and improved upon use with additional data over time. The processor may be that of any general purpose computer or a specific computer for this purpose.

Other aspects of the present disclosure provide a computer readable medium on which is stored a computer program which, when implemented by a computer processor, causes the processor to: perform in silico a three-dimensional structure prediction of a protein using a structure prediction algorithm; identify in silico at least one pair of solvent-exposed amino acids in the protein based on algorithm-predicted factors (e.g., variance in the spatial distance between the two amino acids of the at least one pair; and constrain the structure prediction algorithm using distance measurements collected in vitro between amino acids of the at least one pair of amino acids present in a recombinant copy of the protein using FRET, wherein a FRET donor is attached to one amino acid of the pair and a FRET acceptor is attached to the other amino acid of the pair.

An illustrative implementation of a computer system 1400 that may be used in connection with any of the embodiments of the technology described herein is shown in FIG. 7. The computer system 1400 includes one or more processors 1410 and one or more articles of manufacture that comprise non-transitory computer-readable storage media (e.g., memory 1420 and one or more non-volatile storage media 1430). The processor 1410 may control writing data to and reading data from the memory 1420 and the non-volatile storage device 1430 in any suitable manner, as the aspects of the technology described herein are not limited in this respect. To perform any of the functionality described herein, the processor 1410 may execute one or more processor-executable instructions stored in one or more non-transitory computer-readable storage media (e.g., the memory 1420), which may serve as non-transitory computer-readable storage media storing processor-executable instructions for execution by the processor 1410.

Computing device 1400 may also include a network input/output (I/O) interface 1440 via which the computing device may communicate with other computing devices (e.g., over a network), and may also include one or more user I/O interfaces 1450, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The above-described embodiments can be implemented in any of numerous ways. For example, the embodiments may be implemented using hardware, software or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-discussed functions of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

### Additional Embodiments

Additional embodiments of the present disclosure are encompassed by the following numbered paragraphs.

1. A method comprising:

(i) performing in silico a three-dimensional structure prediction of a protein using a structure prediction algorithm;

(ii) identifying in silico at least one pair of solvent-exposed amino acids in the protein, based on at least one algorithm-predicted factor;

(iii) labeling in vitro the at least one pair of amino acids in at least one recombinant copy of the protein such that a fluorescence resonance energy transfer (FRET) donor is attached to the first amino acid of the pair and a FRET acceptor is attached to the second amino acid of the pair;

(iv) collecting in vitro distance measurements between the two amino acids of the at least one pair using FRET; and

(v) constraining the structure prediction algorithm using the collected distance measurements.

2. The method of paragraph 1, further comprising:

(vi) performing in silico a three-dimensional structure prediction of a protein using the constrained structure prediction algorithm, and optionally further repeating, at least 1, 2, 3, or more times, each of (ii) to (vi).

3. The method of paragraph 1 or 2, wherein the pair of amino acids are separated based on the primary structure of the protein by at least five amino acids.

4. The method of any one of the preceding paragraphs, wherein (i) comprises performing in silico a three-dimensional structure prediction of a protein using a structure prediction algorithm and generating a probabilistic matrix or distogram of the distances between each combination of two amino acids in the protein.

5. The method of any one of the preceding paragraphs, wherein (ii) comprises determining the at least one algorithm-predicted factors for every combination of two solvent-exposed amino acids and rank-ordering every combination based on the factor(s).

6. The method of any one of the preceding paragraphs, wherein the at least one algorithm-predicted factor is:

variance in the spatial distance between the two amino acids of the at least one pair;

the relative importance of the distance between the two amino acids in the structure prediction algorithm; and/or

the structural sensitivity of the pair.

7. The method of paragraph 6, wherein (ii) comprises determining the variance in the spatial distance between every combination of two solvent-exposed amino acids and rank-ordering every combination of two solvent-exposed amino acids based on algorithm-predicted variance in spatial distance, optionally wherein the at least one pair of amino acids is identified as having the largest algorithm-predicted variance in spatial distance.

8. The method of paragraph 6 or 7, wherein, in (ii), the algorithm-predicted variance in the spatial distance between the two amino acids comprises a k-value of between 1 and 100.

9. The method of any one of the preceding paragraphs, wherein the method comprises:

(i) performing in silico a three-dimensional structure prediction of a protein using a structure prediction algorithm;

(ii) identifying in silico 2, 3, 4, 5, or more pairs of solvent-exposed amino acids in the protein based on at least one algorithm-predicted factor;

(iii) labeling in vitro each pair of amino acids in a recombinant copy of the protein such that a fluorescence resonance energy transfer (FRET) donor is attached to the first amino acid of each pair and a FRET acceptor is attached to the second amino acid of each pair, wherein each pair of amino acids is labeled in a different recombinant copy of the protein;

(iv) collecting in vitro distance measurements between the two amino acids of each pair using FRET; and

(v) constraining the structure prediction algorithm using the collected distance measurements.

10. The method of paragraph 9, wherein, in (iii), each different recombinant copy of the protein comprises a unique molecular identifier or barcode sequence.

11. The method of paragraph 9 or 10, wherein, in (iii), each different recombinant copy of the protein is placed into an individual well of a multi-well plate or an individual chamber of a zero-mode waveguide.

12. The method of paragraph 11, wherein each different recombinant copy of the protein is attached to the bottom of an individual well of a multi-well plate or an individual chamber of a zero-mode waveguide, optionally wherein the each different recombinant copy of the protein is attached via a biotin-streptavidin linkage.

13. The method of any one of the preceding paragraphs, wherein one of the amino acids of the at least one pair is a cysteine, a lysine, or a non-natural amino acid, optionally wherein the non-natural amino acid is p-azido-L-phenylalanine.

14. The method of any one of the preceding paragraphs, wherein the FRET acceptor and FRET donor are organic dyes, fluorescent proteins, or quantum dots.

15. The method of paragraph 14, wherein the fluorescent proteins are cyan fluorescent proteins (CFPs) and yellow fluorescent proteins (YFPs); green fluorescent proteins (GFPs) and red fluorescent proteins (RFPs); or far-red fluorescent proteins (FFPs) and infrared fluorescent proteins (IFPs).

16. The method of any one of the preceding paragraphs, wherein the collecting in (iv) involves total internal reflection fluorescence, fluorescence lifetime imaging microscopy, or zero-mode waveguide sensing.

17. The method of any one of the preceding paragraphs, wherein the collecting in (iv) is done using single-molecule methods.

18. The method of any one of the preceding paragraphs, wherein the at least one recombinant copy of the protein is barcoded.

19. The method of paragraph 18, wherein the at least one recombinant copy of the protein is barcoded with a unique molecular identifier, optionally a nucleic acid-based or peptide-based unique molecular identifier.

20. A computer-implemented method comprising:

performing in silico a three-dimensional structure prediction of a protein using a structure prediction algorithm;

identifying in silico at least one pair of solvent-exposed amino acids in the protein based on at least one algorithm-predicted factors; and

constraining the structure prediction algorithm using distance measurements collected in vitro between amino acids of the at least one pair of amino acids present in a recombinant copy of the protein using fluorescence resonance energy transfer (FRET), wherein a FRET donor is attached to one amino acid of the pair and a FRET acceptor is attached to the other amino acid of the pair.

21. The method of paragraph 21, wherein the at least one algorithm-predicted factor is:

variance in the spatial distance between the two amino acids of the at least one pair;

the relative importance of the distance between the two amino acids in the structure prediction algorithm; and/or

the structural sensitivity of the pair.

22. A computer-implemented method comprising:

performing in silico a three-dimensional structure prediction of a protein using a structure prediction algorithm;

identifying in silico at least one pair of solvent-exposed amino acids in the protein based on algorithm-predicted variance in the spatial distance between the two amino acids of the at least one pair; and

constraining the structure prediction algorithm using distance measurements collected in vitro between amino acids of the at least one pair of amino acids present in a recombinant copy of the protein using fluorescence resonance energy transfer (FRET), wherein a FRET donor is attached to one amino acid of the pair and a FRET acceptor is attached to the other amino acid of the pair.

23. A computer readable medium on which is stored a computer program which, when implemented by a computer processor, causes the processor to:

perform in silico a three-dimensional structure prediction of a protein using a structure prediction algorithm;

identify in silico at least one pair of solvent-exposed amino acids in the protein based on at least one algorithm-predicted factor; and

constrain the structure prediction algorithm using distance measurements collected in vitro between amino acids of the at least one pair of amino acids present in a recombinant copy of the protein using fluorescence resonance energy transfer (FRET), wherein a FRET donor is attached to one amino acid of the pair and a FRET acceptor is attached to the other amino acid of the pair.

24. The method of paragraph 23, wherein the at least one algorithm-predicted factor is:

variance in the spatial distance between the two amino acids of the at least one pair;

the relative importance of the distance between the two amino acids in the structure prediction algorithm; and/or

the structural sensitivity of the pair.

25. A computer readable medium on which is stored a computer program which, when implemented by a computer processor, causes the processor to:

perform in silico a three-dimensional structure prediction of a protein using a structure prediction algorithm;

identify in silico at least one pair of solvent-exposed amino acids in the protein based on algorithm-predicted variance in the spatial distance between the two amino acids of the at least one pair; and

constrain the structure prediction algorithm using distance measurements collected in vitro between amino acids of the at least one pair of amino acids present in a recombinant copy of the protein using fluorescence resonance energy transfer (FRET), wherein a FRET donor is attached to one amino acid of the pair and a FRET acceptor is attached to the other amino acid of the pair.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean ±10% of the recited numerical value.

Where a range of values is provided, each value between the upper and lower ends of the range are specifically contemplated and described herein.

What is claimed is:

1. A method comprising:
(i) performing in silico a three-dimensional structure prediction of a protein using a structure prediction algorithm;
(ii) identifying in silico at least one pair of solvent-exposed amino acids in the protein, based on at least one algorithm-predicted factor;
(iii) labeling in vitro the at least one pair of amino acids in at least one recombinant copy of the protein such that a fluorescence resonance energy transfer (FRET) donor is attached to the first amino acid of the pair and a FRET acceptor is attached to the second amino acid of the pair;
(iv) collecting in vitro distance measurements between the two amino acids of the at least one pair using FRET; and
(v) constraining the structure prediction algorithm using the collected distance measurements.

2. The method of claim 1, further comprising:
(vi) performing in silico a three-dimensional structure prediction of a protein using the constrained structure prediction algorithm, and optionally further repeating, at least 1, 2, 3, or more times, each of (ii) to (vi).

3. The method of claim 1, wherein the pair of amino acids are separated based on the primary structure of the protein by at least five amino acids.

4. The method of claim 1, wherein (i) comprises performing in silico a three-dimensional structure prediction of a protein using a structure prediction algorithm and generating a probabilistic matrix or distogram of the distances between each combination of two amino acids in the protein.

5. The method of claim 1, wherein (ii) comprises determining the at least one algorithm-predicted factors for every combination of two solvent-exposed amino acids and rank-ordering every combination based on the factor(s).

6. The method of claim 1, wherein the at least one algorithm-predicted factor is:
variance in the spatial distance between the two amino acids of the at least one pair;
the relative importance of the distance between the two amino acids in the structure prediction algorithm; and/or
the structural sensitivity of the pair.

7. The method of claim 6, wherein (ii) comprises determining the variance in the spatial distance between every combination of two solvent-exposed amino acids and rank-ordering every combination of two solvent-exposed amino acids based on algorithm-predicted variance in spatial distance, optionally wherein the at least one pair of amino acids is identified as having the largest algorithm-predicted variance in spatial distance.

8. The method of claim 6, wherein, in (ii), the algorithm-predicted variance in the spatial distance between the two amino acids comprises a k-value of between 1 and 100.

9. The method of claim 1, wherein the method comprises:
(i) performing in silico a three-dimensional structure prediction of a protein using a structure prediction algorithm;
(ii) identifying in silico 2, 3, 4, 5, or more pairs of solvent-exposed amino acids in the protein based on at least one algorithm-predicted factor;
(iii) labeling in vitro each pair of amino acids in a recombinant copy of the protein such that a fluorescence resonance energy transfer (FRET) donor is attached to the first amino acid of each pair and a FRET acceptor is attached to the second amino acid of each pair, wherein each pair of amino acids is labeled in a different recombinant copy of the protein;
(iv) collecting in vitro distance measurements between the two amino acids of each pair using FRET; and
(v) constraining the structure prediction algorithm using the collected distance measurements.

10. The method of claim 9, wherein, in (iii), each different recombinant copy of the protein comprises a unique molecular identifier or barcode sequence.

11. The method of claim 9, wherein, in (iii), each different recombinant copy of the protein is placed into an individual well of a multi-well plate or an individual chamber of a zero-mode waveguide.

12. The method of claim 11, wherein each different recombinant copy of the protein is attached to the bottom of an individual well of a multi-well plate or an individual chamber of a zero-mode waveguide, optionally wherein the each different recombinant copy of the protein is attached via a biotin-streptavidin linkage.

13. The method of claim 1, wherein one of the amino acids of the at least one pair is a cysteine, a lysine, or a non-natural amino acid, optionally wherein the non-natural amino acid is p-azido-L-phenylalanine.

14. The method of claim 1, wherein the FRET acceptor and FRET donor are organic dyes, fluorescent proteins, or quantum dots, optionally wherein the fluorescent proteins are cyan fluorescent proteins (CFPs) and yellow fluorescent proteins (YFPs); green fluorescent proteins (GFPs) and red fluorescent proteins (RFPs); or far-red fluorescent proteins (FFPs) and infrared fluorescent proteins (IFPs).

15. The method of claim 1, wherein the collecting in (iv) involves total internal reflection fluorescence, fluorescence lifetime imaging microscopy, zero-mode waveguide sensing, and/or single-molecule methods.

16. The method of claim 1, wherein the at least one recombinant copy of the protein is barcoded, optionally wherein the at least one recombinant copy of the protein is barcoded with a unique molecular identifier, optionally a nucleic acid-based or peptide-based unique molecular identifier.

17. A computer-implemented method comprising:
performing in silico a three-dimensional structure prediction of a protein using a structure prediction algorithm;
identifying in silico at least one pair of solvent-exposed amino acids in the protein based on at least one algorithm-predicted factors; and
constraining the structure prediction algorithm using distance measurements collected in vitro between amino acids of the at least one pair of amino acids present in a recombinant copy of the protein using fluorescence resonance energy transfer (FRET), wherein a FRET donor is attached to one amino acid of the pair and a FRET acceptor is attached to the other amino acid of the pair.

18. The computer-implemented method of claim 17, wherein the at least one algorithm-predicted factors are algorithm-predicted variance in the spatial distance between the two amino acids of the at least one pair.

19. A computer readable medium on which is stored a computer program which, when implemented by a computer processor, causes the processor to:
perform in silico a three-dimensional structure prediction of a protein using a structure prediction algorithm;
identify in silico at least one pair of solvent-exposed amino acids in the protein based on at least one algorithm-predicted factor; and
constrain the structure prediction algorithm using distance measurements collected in vitro between amino acids of the at least one pair of amino acids present in a recombinant copy of the protein using fluorescence resonance energy transfer (FRET), wherein a FRET donor is attached to one amino acid of the pair and a FRET acceptor is attached to the other amino acid of the pair.

20. The computer readable medium of claim 19, wherein the at least one algorithm-predicted factor is algorithm-predicted variance in the spatial distance between the two amino acids of the at least one pair.
